(19) United States
(12) Patent Application Publication
(10) Pub. No.: US 2017/0319746 A1
(43) Pub. Date: Nov. 9, 2017

(54) A METHOD FOR BUILDING A STRUCTURE CONTAINING LIVING CELLS

(71) Applicant: ECOLE POLYTECHNIQUE FEDERALE DE LAUSANNE (EPFL), Lausanne (CH)

(72) Inventors: Matthias Lutolf, Tolochenaz (CH); Andrea Negro, Gollion (CH)

(21) Appl. No.: 15/535,322

(22) PCT Filed: Dec. 12, 2014
(86) PCT No.: PCT/EP2014/077648

§ 371 Date: Jun. 12, 2017

(51) Int. Cl. A61L 27/38; C12P 19/04; B33Y 70/00; B33Y 80/00; B29C 64/40; A61L 27/26; A61L 27/48; A61L 27/52; A61L 27/54; A61L 27/58; C08J 3/075; C09D 105/04; C09D 171/02; C12N 9/10; C12N 5/077; C12N 5/00; C12M 1/00; C12M 3/06; C12M 3/00; B33Y 10/00; B29K 71/00; B29C 64/112; B29K 105/00

(52) U.S. Cl. CPC A61L 27/3804; C12Y 402/02003; B33Y 10/00; C12P 19/04; C12Y 203/02013; B33Y 70/00; B33Y 80/00; B29C 64/40; A61L 27/26; A61L 27/48; A61L 27/52; A61L 27/54; A61L 27/58; C08J 3/075; C09D 105/04; C09D 171/02; C12N 9/1044; C12N 5/0656; C12N 5/0062; C12M 29/10; C12M 23/16; C12M 21/08; C08J 2371/02; A61L 2300/414; A61L 2400/18; A61L 2430/00; B29K 2071/02; C12N 2537/10; B29K 2995/0056; B29C 64/112; C08J 2305/04; C12N 2513/00; C12N 2533/74; B29K 2105/0061

(57) ABSTRACT

A composition comprising a first material and a second material, wherein said first material is cross-linkable by a first cross-linking reaction and said second material is cross-linkable by a second cross-linking reaction, wherein said first cross-linking reaction and said second cross-linking reaction are inducible by a common activator.

The present invention relates to a composition comprising a first and second material, wherein said first material is cross-linkable by a first cross-linking reaction and said second material is cross-linkable by a second cross-linking reaction, wherein said first cross-linking reaction and said second cross-linking reaction are inducible by a common activator. The present invention also relates to a bioink comprising such a composition, a hydrogel formed from such a composition and a structure formed from such a composition or hydrogel. The invention also relates to a method of making a structure comprising such a hydrogel, a structure containing living cells produced by the same method, an artificial tissue produced by such a method, and a method for aligning and synchronizing multiple dispensing units, according to the independent claims.

Bioprinting approaches have come to the fore because of their huge potential to pattern biomaterials and cells into living 3D constructs that resemble tissues and could find widespread applications in drug discovery and regenerative medicine. Owing to its high resolution and biocompatibility, inkjet printing is particularly interesting in this context. Although hydrogels have already been identified as a preferential materials class for inkjet printing, an adequate hydrogel bioink has yet to be developed. The ideal bioink would have distinct physico-chemical properties in the liquid state, very rapidly transform into a solid hydrogel upon dispensing, and also possess the necessary bioactive characteristics to guide cell development into a functional tissue.

The design of bioinks for bioprinting entails multiple technical and biological challenges. Since hydrogels are biophysically similar to native extracellular matrices (ECM) in our tissues, they can be considered as ideal bioink candidates for building up 3D structures. However, the design of a functional hydrogel bioink must also take into consideration the entire printing and cell growth process that leads the formation of a tissue. First, the bioink must fulfill certain fluid mechanics requirements towards optimal droplet generation. Furthermore, proper 3D droplet packing upon impact must also be taken into account. In essence, the liquid to solid transformation (i.e. the bioink crosslinking) has to be fast enough to “freeze” the drop so that it retains a 3D profile. Such fast reaction rates also allow one layer to be printed over the previous one, maintaining the rapid-prototyping mode of building 3D objects. In addition to these technical bioink requirements, it is crucial that bioinks in their liquid form are fully cell-compatible, and that the cross-linking reaction takes place under physiological conditions. This minimizes the amount of stress that is exerted on cells exposed to the bioink. After 3D printing, the bioactivity of the cross-linked hydrogel bioink plays an essential role. The 3D matrix should provide instructive cues for cells, such as tissue-specific adhesion ligands and growth factors, which help in guiding cellular self-organization into tissue-like structures. Finally, of equal importance is the degradability of the hydro-gel bioink. Ideally, cells should be able to degrade their artificial microenvironment and replace it with their own.

Tissues are multi-component entities harboring various cell types that are embedded in cell- and tissue-specific extracellular matrices. These matrices have a characteristic three-dimensional histological architecture that is crucial for tissue function. Furthermore, tissues are dynamic entities in which cells self-organize by migrating, proliferating, specializing and actively remodeling their extracellular matrices.

Recapitulating spatial and temporal tissue complexity outside of the body, to an extent that allows capturing some of the functionality of a tissue, represents one of the most exciting challenges in modern tissue engineering. Replicating this complexity in-vivo entails two key aspects: Firstly, formulating biomaterials that are able to mimic the physiological extracellular milieu, and as such promote physiological cell behaviors and cellular cell organization as in tissues. Secondly, as cell organization and morphogenesis are limited in in-vitro culture systems, it may be necessary to arrange tissue building blocks in an in-vivo like three-dimensional manner.

Ink jet printing platforms are highly efficient tools for recreating the three-dimensional architecture of a tissue, using computer aided deposition. Drop on demand systems, in particular, grant the possibility of depositing a wide range of soft biomaterials in successive layers to generate three-dimensional structures.

Pataky et al. have reported the microdrop printing of so-called hydrogel bioinks into three dimensional tissue-like geometries (Adv. Mater. 2012, 24, 391-396). Generally, in order to print microscopic structures in a layer-by-layer fashion from small microdroplets, the dispensed droplets must retain their three-dimensional structure to some extent. To this end, their system relied on a printing setup composed of a hydrated gelatin substrate acting as a Ca²⁺ reservoir, from which Ca²⁺ ions diffused upwards into the printed alginate-containing droplets to induce rapid gelation. Although this technology allowed for the rapid formation of tissue-like structures, it had been limited to single component bio printing. Furthermore, the construction of “overhanging” structural motifs, as usually found in cavities, was very limited.

Kolesky et al. disclosed a method for fabricating engineered tissue-like constructs replete with vasculature, multiple types of cells and extracellular matrices (Adv. Mater. 2014, 26, 3124-3130). To this end, they constructed a 3D bio-printer with four independently controlled print-heads. In order to create hollow cavities, a specialized fugitive ink was used that becomes water soluble upon cooling below a certain temperature. To create the solid parts and the extracellular matrix, a photo polymerizable gelatin methacrylate was used as bulk matrix and cell carrier. By taking advantage of this complementary behavior, three-dimensional vascular networks were printed. In order to introduce endothelial cells, lining the vascular walls and providing a barrier to fluid diffusion, while simultaneously facilitating homeostatic functions and helping to establish vascular niches specific to various tissues, the bifurcated vascular networks produced were injected with a human vein endothelial cell suspension to obtain a nearly confluent layer. Furthermore, engineered tissue-like constructs with multiple types of cells were fabricated. Although this approach allowed for the generation of vascularized heterogeneous tissue constructs based on bio printing, there was the drawback that the endothelial cells had to be introduced into the system in an extra step. Moreover, the system suffered from a limited printing resolution with tube diameters in the printed structures of about 1 mm. Physiologically relevant microvessels, such as capillaries, could therefore not be produced.

The patent application US 2011/0212501 A1 describes three-dimensional multi-layered tissue-like hydrogel structures and methods for making same. The methods also relied on the drop-on-demand printing of cross-linkable materials. In some embodiments, the three-dimensional multilayer hydrogel constructs formed further comprised channels. These channels could be perfused with fluids such as culture media, plasma, artificial blood or blood, in order to nourish cells in the constructs. The formation of channels or voids also relied on the removal of sacrificial material. However, the method relied on the deposition of hydrogel precursor materials followed by the application of nebulized cross-linking materials. This made the method rather unreliable and cumbersome. Moreover, the system provided a rather limited printing resolution. Physiologically highly relevant microvessels, the smallest systems of blood vessels in the body, could thus for example not be produced.

It is a problem underlying the present invention to overcome the drawbacks in the prior art. In particular, it is a problem underlying the present invention to provide an improved composition that is suitable for applications such as bioprinting. It is also a problem underlying the invention to provide an improved method for building a structure, optionally containing living cells, especially by 3D bio-printing techniques. The method should allow for multicomponent printing of different kinds of extracellular matrices or cell types. It should be useful in various application fields and enable high resolution printing. The method should be rapid, cost-efficient and should allow for the creation of complex tissue-like structures. In this context, it is also a problem underlying the present invention to provide hybrid hydrogel compositions for use in such a method. Furthermore, it is a problem underlying the present invention to provide an improved method for aligning and synchronizing multiple dispensing units. Moreover, it is a problem underlying the present invention to provide structures containing living cells, wherein these structures have improved properties.

These problems are solved by the methods, structures and compositions according to the independent claims.

### SUMMARY OF INVENTION

Thus viewed from a first aspect, the present invention provides a composition comprising a first material and a second material, wherein said first material is cross-linkable by a first cross-linking reaction and said second material is cross-linkable by a second cross-linking reaction, wherein said first cross-linking reaction and said second cross-linking reaction are inducible by a common activator.

Viewed from a further aspect, the invention provides a bioink comprising a composition as hereinbefore described.

Viewed from a further aspect, the present invention provides a hydrogel formed from the composition as hereinbefore described, or a bioink as hereinbefore described.

Viewed from a further aspect, the present invention provides a hydrogel comprising a cross-linked first material formed by a first cross-linking reaction and a second cross-linked material formed by a second cross-linking reaction, wherein said first cross-linking reaction and said second cross-linking reaction are inducible by a common activator.

Viewed from a further aspect, the present invention provides a structure formed from a composition as hereinbefore described or a bioink as hereinbefore described.

Viewed from a further aspect, the present invention provides a structure comprising a hydrogel as hereinbefore described.

Viewed from a further aspect, the present invention provides a method of making a structure as hereinbefore described, comprising depositing a composition as hereinbefore described or a bio-ink as hereinbefore described on a substrate, wherein said substrate provides a common activator for inducing said first cross-linking reaction in said first composition and said second cross-linking reaction in said second composition.

Viewed from a further aspect, the present invention provides a method of making a structure, comprising the steps of:
i. forming at least one, preferably a plurality of, sacrificial layer(s) on a substrate;
ii. forming at least one, preferably a plurality of, permavent layer(s) on said substrate or said first sacrificial layer(s); and

wherein said at least one sacrificial layer is derived from a first composition and said at least one permanent layer is derived from at least one second composition; and

wherein said at least one sacrificial layer is formed by a first cross-linking reaction of said first composition and said at least one permanent layer is formed by a second cross-linking reaction of said second composition;

wherein said first cross-linking reaction and said second cross-linking reaction are induced by said common activator.

Viewed from a further aspect, the present invention provides a structure produced by a method as hereinbefore described.

Viewed from a further aspect, the present invention provides a method for aligning and synchronizing multiple dispensing units, wherein a multi-component test-pattern is deposited and assessed.

Viewed from a further aspect, the present invention provides a method of providing an artificial tissue using a structure as hereinbefore described as a template.

Viewed from a further aspect, the present invention provides an artificial tissue produced by a method as hereinbefore described.

### BRIEF DESCRIPTION OF THE DRAWINGS

FIGS. 1a-d: Schematic representation of a method for building a structure containing living cells by droplet-by-droplet deposition according to the present invention;

FIG. 2: flow chart of a method for aligning and synchronizing multiple dispensing units according to the present invention;

FIG. 3: ejection calibration process in an aligning- and synchronizing method according to the present invention;

FIG. 4: schematic representation of the alignment patterning step in an aligning- and synchronizing netted according to the present invention;

FIG. 5: schematic representation of the linear interpolation step in an aligning- and synchronizing method according to the present invention;

FIG. 6: multi-component test-pattern with linear interpolation in an aligning- and synchronizing method according to the present invention;

FIG. 7: test-pattern printed upon achievement of proper alignment by a method according to the present invention;

FIG. 8: perfusion chamber for a network of perfusable channels according to the present invention;

FIG. 9: perfusion systems of a perfusion chamber for a network of perfusable channels according to the present invention;

FIG. 10: cross-section of a perfusion chamber according to FIG. 9;

FIG. 11: partial enlargement of a cross-section according to FIG. 10;

FIG. 12: schematic representation of the formation of a hybrid hydrogel mixture;

FIG. 13: schematic representation of dynamic control of gel properties to enhance three-dimensional cellular responses;

FIG. 14: schematic representation of modularity and combinatorial preparation of hybrid hydrogel compositions;

FIGS. 15a-e: schematic representation of the application of the alginate-PEG-based hybrid hydrogel system in droplet deposition;

FIG. 16: network of perfusable channels according to the present invention;

FIG. 17: cell-proliferation in a network of perfusable channels according to the present invention.

### DESCRIPTION OF THE INVENTION

The compositions of the present invention comprise a first material and a second material, wherein the first material is cross-linkable by a first cross-linking reaction and the second material is cross-linkable by a second cross-linking reaction, wherein the first cross-linking reaction and the second cross-linking reaction are inducible by a common activator.

The first and the second cross-linking reactions are substantially compatible with each other. In the present context, the phrase “substantially compatible with each other” relates to two or more reactions, for example chemical reactions, that can proceed simultaneously in the same environment without leading to a substantially different product-distribution or having any other substantial adverse effect on each other.

Preferably the first material is a polymer, more preferably a biopolymer. In this context, the term biopolymer refers to a polypeptide, a polysaccharide or a polynucleotide. More preferably, the first material is a polysaccharide, still more preferably a polysaccharide derived from alginate. In this context, the term alginate refers to a natural polysaccharide that can be extracted from seaweeds. It is characterized by a linear sequence of the two monomers of (1-4)-linked β-D-mannuronate (M residue) and C-5 epimer α-L-guluronate (G residue). The alginate chains consist of blocks of G-monomers (G-blocks) that are characterized by a regular geometry and lead to an accumulation of negative charges.

In preferred compositions the second material comprises a biocompatible synthetic or semi-synthetic polymer. Preferably the second material comprises a hydrophilic polymer. Preferably the second material comprises a swellable polymer. Preferably the second material comprises a copolymer of a hydrophilic polymer and an oligopeptide. Preferably the oligopeptide is or provides a substrate for said second cross-linking reaction. Preferably the oligopeptide also is or provides a substrate for at least one additional reaction, e.g. an enzymatic reaction.

More preferably the second material comprises a modified or unmodified polyglycol, still more preferably a modified or unmodified polyethylene glycol, e.g. a branched or unbranched modified or unmodified polyethylene glycol. Polyethylene glycol (PEG) is a synthetic and highly hydrophilic polymer that can be modified to be cross-linked enzymatically. Moreover, if desired, it is possible to tether a variety of bioactive molecules into the compositions by modifying the polyethylene glycol in the composition. Thus in preferred compositions, the second material further comprises an additional biomolecule having a biological function, preferably a signalling molecule e.g. a cell-adhesive peptide or a growth factor. Examples of such biomolecules include, but are not limited to, a cell-adhesive peptide such as RDG or a growth factor such as VEGF.

Examples of preferred second materials are described in Biomacromolecules 2007, 8, 3000-3007.

In preferred compositions, the first material, when cross-linked by the first cross-linking reaction, is degradable and the second material, when cross-linked by the second cross-linking reaction, is not degradable. In other words the first material, when cross-linked, can be degraded in the presence of the second material, when cross-linked, with no or substantially no degradation of this second material.

In preferred compositions, the common activator is a chemical or biological agent, or a physical stimulus. In the present context, the term “activator” refers to any chemical or biological species, or physical stimulus, that can cause a reaction to commence. A chemical activator can be a catalyst, an acid, a base or a metal salt or ion. A chemical activator can also be an enzyme cofactor. A biological activator can be an enzyme. A physical activator can be heat or UV-irradiation.

Preferably the common activator is a chemical agent, such as an organic compound, a metal salt or ion, an acid or a base. More preferably the common activator is a metal ion, preferably an alkali metal ion or an alkaline earth metal ion. Still more preferably, the common activator is a calcium ion, e.g Ca²⁺.

When Ca²⁺ is added to an alginate solution, it rapidly interacts with two different G-blocks, resulting in the generation of crosslinks that ultimately result in hydrogel formation.

Polyethylene glycol can be cross-linked enzymatically, for example by a transglutaminase, such as FXIIIa, a key enzyme acting in the blood coagulation cascade to crosslink fibrinogen into fibrin gels. Transglutaminases are active only in the presence of Ca²⁺. When this cofactor is not present, the enzyme cannot perform the reaction. As such, the calcium-mediated action of transglutaminase leads to the cross-linking of a polymer into a hydrogel network.

In preferred compositions, the first cross-linking reaction and the second cross-linking reaction may by the same reaction or are different reactions. Preferably the first the first cross-linking reaction and the second cross-linking reaction are different reactions. In some preferred compositions the first cross-linking reaction and the second cross-linking reaction occur independently of each other.

In preferred compositions, the first cross-linking reaction is relatively fast, preferably in the order of about 0.1 to 10 seconds e.g. about 1 second, and the second cross-linking reaction is relatively slow, preferably in the order of about 10 to 60 minutes, e.g. about 15 to 30 minutes.

Preferred compositions further comprise a buffer, preferably tris(hydroxymethyl)aminomethane buffer. The compositions may preferably comprise a surfactant, preferably a nonionic propylene glycol-derived surfactant. Preferred surfactants are available under the trade name Pluronic.

Further preferred compositions comprise an enzyme, preferably a cross-linking enzyme. As described above, a preferred enzyme present in the compositions is a transglutaminase, such as FXIIIa. FXIIIa can enzymatically cross-link polyethylene glycol.

Transglutaminases are active only in the presence of Ca²⁺. When this activator is not present, the enzyme cannot perform the reaction. As such, the calcium-mediated action of transglutaminase leads to the cross-linking of a polymer, e.g. a polyethylene glycol.

It is desirable that the compositions described above (e.g. comprising alginate and PEG) can be kept in a liquid state for a prolonged period of time. This allows storing of the solutions by providing them with a reasonable shelf-life. This in turn means that the compositions described can be employed in a drop-let-by-droplet deposition device (3D printer). However, in the previously described compositions, residual amounts of calcium can lead to cross-linking within a few minutes upon preparation. This can lead to cross-linking within a dispensing unit, which causes clogging and avoids proper deposition. In the present case, the main source of such deleterious calcium is the FXIIIa stock solution. During the activation of fibrogammin (the inactive form of FXIIIa), calcium is present in the buffer solution used to dilute thrombin, the enzyme that activates the fibrogammin. The thrombin solution contains 22.5 millimoles per liter of calcium, which is later diluted by a factor of 10, when added to the fibrogammin solution. This results in a final calcium concentration of 2.25 millimoles per liter.

Thus in preferred compositions, a chelating agent is present. Preferably, the chelating agent is a biocompatible chelating agent, in particular ethylenediaminetetraacetate (EDTA) or citric acid. The chelating agent, added in proper concentrations, sequesters calcium ions and the cross-linking reaction is supressed until exposure to extrinsic calcium sources. Such calcium sources can be calcium solutions or calcium releasing solid substrates. Preferably, the chelating agent concentration does not affect the cross-linking kinetics of the composition. That is, when positively charged ions are provided, the first cross-linking reaction and the second cross-linking reaction occur with no further delays. Furthermore, the chelating agent preferably does not affect cell viability when cells are dispersed in the composition.

Preferably the first cross-linking reaction and the second cross-linking reaction proceed under mutually compatible reaction conditions, e.g. physiological conditions. In other words, the reaction conditions required to induce the first cross-linking reaction are similar to, or at least compatible with, the reaction conditions required to induce the second cross-linking reaction.

Preferred compositions comprise a polysaccharide derived from alginate, a modified or unmodified polyethylene glycol and a transglutaminase. Preferably the first material, e.g alginate, is present in the composition in about 0.3 to 1.0% w/v more preferably about 0.5% w/v. Preferably the second material, e.g. polyethylene glycol, is present in the composition in about 2.0 to 3.5% w/v, more preferably about 2.5% w/v.

The compositions of the invention can be used as a bioink in bioprinting applications. Thus another aspect of the invention is a bioink comprising a composition as hereinbefore described. Preferably the bioink further comprises cells, preferably mammalian cells.

Generally, cross-linking of the first and second materials described above can be triggered by exposure to the common activator, e.g. calcium ions, in two ways: 1) a calcium containing buffer is added to the precursor mixture or 2) calcium is delivered to a precursor solution via diffusion from a solid substrate that stores the ion. This way, three-dimensional structures of hybrid hydrogels can be fabricated from a substrate in an additive manner. Additional biologically active components, such as living cells or extra cellular matrix components can be added to the mixture. If desired, these additional components can be tethered into the forming hydrogel matrix, either covalently or via affinity-binding interactions.

Thus a further aspect of the invention is a hydrogel formed from a composition as hereinbefore described, or a bioink as hereinbefore described.

A further aspect of the invention is a hydrogel comprising a cross-linked first material formed by a first cross-linking reaction and a second cross-linked material formed by a second cross-linking reaction, wherein the first cross-linking reaction and the second cross-linking reaction are inducible by a common activator.

Preferably in the hydrogel the cross-linked first material is a polypeptide, a polysaccharide or a polynucleotide, more preferably a polysaccharide, e.g. a polysaccharide derived from alginate.

Preferably the cross-linked second material comprises a bio-compatible synthetic or semi-synthetic polymer, preferably a modified or unmodified polyethylene glycol. Preferably the second material comprises a co-polymer of a hydrophilic polymer and an oligopeptide, preferably an oligopeptide that is or provides a substrate for the second cross-linking reaction.

In preferred hydrogels, the second material comprises a copolymer of a hydrophilic polymer and an oligopeptide and the oligopeptide is or provides a substrate for at least one additional reaction, e.g. an enzymatic reaction. In some preferred hydrogels, the second material further comprises an additional biomolecule having a biological function, preferably a signalling molecule e.g. a cell-adhesive peptide or a growth factor.

In the above-mentioned hydrogels, the first cross-linked material can be derived from alginate. Furthermore, the second cross-linked material can be a copolymer of polyethylene glycol (PEG) and an oligopeptide and the second cross-linking reaction can preferably be mediated by a cross-linking enzyme, in particular by a transglutaminase. These two independent hydrogel systems share calcium (Ca²⁺) as a common entity enabling cross-linking. The common activator can be calcium ions (Ca²⁺).

In preferred hydrogels, the cross-linked second material is selectively degradable by a cell-controlled mechanism. In this context, a cell-controlled mechanism refers to a process which is under control of cells within the hydrogel; for example, such as occurs in natural extracellular matrix degradation and remodelling by cells within the ECM in vivo. Preferably this cell-controlled mechanism is the enzymatic degradation of the cross-linked second material, i.e. the cross-linked second material is selectively degradable using an enzyme, preferably a cell-secreted protease, e.g. a matrix metalloprotease. In this way, the cross-linked second material can be degraded by cells in such a way that cells can remodel the hydrogel and develop into a tissue, in analogy to the extracellular matrix of tissues.

Hybrid hydrogel networks of the above-mentioned kind constitute adaptable matrix compositions being capable of dynamically controlling the behavior of various cell types, in particular mammalian cell types. The hybrid hydrogel networks are able to synergistically interact in order to generate a plurality of cell specific micro environments.

Such hybrid hydrogels allow tailoring the network composition in order to mimic physiological cell environments by adding bioactive moieties and/or modifying the physical hydrogel characteristics. In particular, the tailoring of the properties of the hybrid hydrogel networks can be achieved by the combinatorial nature of the system, whereby the solid content and architecture of both polymer networks as well as additional components can be independently varied. Such a hybrid hydrogel network permits a highly modular two-step degradation process for dynamic control of the mechanical properties of the hybrid biomaterial. Furthermore, the hybrid hydrogel network allows for dynamic control of cellular environments by selective removal of one of the polymer network(s) to provide a more permissive microenvironment to the hosted cells via the generation of gel defects. A hybrid hydrogel network of the above mentioned kind has versatile applications in cell biology, developmental biology, stem cell biotechnology, drug discovery, disease modeling, pharmaceutical development, tissue engineering and regenerative medicine. Hence, hybrid hydrogels can be exploited in additive manufacturing applications towards biologically relevant applications.

Thus in preferred embodiments, the cross-linked first material of the hydrogel is selectively removable, e.g. degradable, in the presence of said cross-linked second material. Preferably the cross-linked first material is selectively degradable in a biocompatible process, e.g. using an enzyme such as alginate lyase. In this context, the term biocompatible process refers to a a process which is permits survival and maintenance of cells disposed in the hydrogel, either during the process, and/or after the process has taken place. For example, degradation of the first material by means of an enzymatic process using alginate lyase is biocompatible, since cells may be maintained in the hydrogel after such degradation. In this context, selectively removing the cross-linked first material means that the cross-linked first material is removed from the structure without substantially affecting the cross-linked second material. Selective removal can be achieved by various extrinsic treatments, such as dissolving in a suitable solvent or chemical or biological degradation. Preferably the selective removal is a biocompatible process. In preferred embodiments, an enzyme, more preferably a lyase, is used to selectively remove the cross-linked first material. Particularly preferably, alginate lyase is used to selectively remove the cross-linked second material.

A further aspect of the invention is a structure formed from a composition as hereinbefore defined or a bioink as hereinbefore defined.

Another aspect of the invention is a structure comprising a hydrogel as hereinbefore described. Preferably the structure further comprises living cells, preferably living mammalian cells. Preferably the living cells are present in the first composition or the cross-linked first composition.

A further aspect of the invention is a method of making a structure as hereinbefore described, comprising depositing a composition as hereinbefore described or a bioink as hereinbefore described on a substrate, wherein the substrate provides a common activator for inducing the first cross-linking reaction in the first composition and the second cross-linking reaction in the second composition. Preferably the common activator is as hereinbefore described, e.g. most preferably calcium (Ca²⁺) ions.

A yet further aspect of the invention is a method of making a structure, comprising the steps of:
i. forming at least one, preferably a plurality of, sacrificial layer(s) on a substrate; and
ii. forming at least one, preferably a plurality of, permanent layer(s) on said substrate or said first sacrificial layer(s);
   wherein the at least one sacrificial layer is derived from a first composition and the at least one permanent layer is derived from at least one second composition; and
   wherein the at least one sacrificial layer is formed by a first cross-linking reaction of the first composition and the at least one permanent layer is formed by a second cross-linking reaction of the second composition;
   wherein the first cross-linking reaction and the second cross-linking reaction are induced by the common activator. Preferably the common activator is as hereinbefore described, e.g. most preferably calcium (Ca²⁺) ions.

Preferably the first sacrificial layer(s) is deposited directly on the substrate. In this context, direct deposition of a layer on the substrate layer means that no other layer has previously been deposited on the substrate layer and that the directly deposited layer has immediate contact with the substrate layer. On the other hand, indirect deposition of a layer on the substrate layer means that at least one other layer has previously been deposited on the substrate layer and the indirectly deposited layer has no immediate contact to the substrate layer.

In preferred methods the substrate is a source of calcium ions (Ca2+) and the sacrificial layer(s) can be derived from an alginate-containing first composition. By way of example, the substrate can be a gelatin plate containing high concentrations of a calcium salt, in particular calcium chloride (CaCl₂). With such a substrate, macroscopic structures can be printed in a layer-by-layer fashion from small micro droplets by the deposition of alginate-containing compositions. The hydrated gelatin substrate acts as a calcium ion-reservoir, from which calcium (Ca²⁺) diffuses upwards into the printed droplets to induce gelation.

In preferred methods the at least one sacrificial layer is degradable by a reaction that does not degrade the at least one permanent layer.

Preferably the method further comprises the step of selectively removing, in particular degrading, the at least one sacrificial layer to obtain at least one hollow space.

Preferably, the selective removal, e.g. degradation, of the at least one sacrificial layer can be effected in a biocompatible process. Preferably the selective removal is carried out enzymatically, i.e. using an enzyme. In particular, selective removal, e.g. degradation, of the at least one sacrificial layer can be effected by an alginate lyase. An enzymatic degradation usually shows a very high degree of selectivity, which allows for the effective removal of the sacrificial layers with minimal damage of the permanent layers.

In a preferred method, the first composition comprises living cells. Preferably the living cells are eukaryotic, preferably mammalian cells. However, the method is not restricted to this class of cells and any other kind of cell belonging to a multicellular organism can be employed in this method. Preferably upon removal, e.g. degradation, of the sacrificial layer(s), the living cells are liberated and adhere to (an) inner or outer surface(s) of the permanent layers, preferably in a non-uniform distribution.

By using this method, a three dimensional tissue-like structure, comprising hollow spaces corresponding to vasculature, can be created efficiently. Notably, no extra step is required to introduce cells, in particular endothelial cells, that adhere to the permanent layers. Furthermore, as upon removal of the sacrificial layer, the liberated cells can undergo controlled sedimentation, a non-uniform cellular distribution can be achieved in the hollow spaces.

In preferred methods, at least one of the sacrificial layers and permanent layers is deposited by extrusion or by printing. Preferably, at least one of the sacrificial layers and permanent layers is deposited by printing, particularly droplet-by-droplet deposition, preferably by a thermal or piezoelectric ink jet technique. Droplet by droplet deposition provides a highly efficient tool to create complex three-dimensional tissue-like architectures in high efficiency and with several-fold higher resolution than other 3D bio-printing methods.

Furthermore, living cells can additionally form part of the second composition(s). In such applications, cells of a desired type can be introduced into a bulk extracellular matrix together with endothelial cells, for example, that cover the vasculature-like cavities. This way, a tissue-like perfusable structure can be created.

The depositing of the sacrificial layers and of the permanent layers can be effected by multiple dispensing units that are aligned and synchronized to one another. The presence of multiple dispensing units is a requirement for multi-component deposition. In order to achieve high resolution in the deposition process, it is a requirement that the dispensing units are aligned and synchronized to each other.

In preferred methods, the at least one hollow space can form a network, in particular a microfluidic network, of perfusable channels. In this context, the term “microfluidic network” refers to a network of channels with a diameter of less than 1 mm. Such a perfusable network, in particular a microfluidic network, plays a crucial role in imparting, supporting or sustaining the biometric function of an engineered tissue. Without the proximity to a perfused microvasculature, providing essential nutrients, growth- and signal factors and waste transport, most cells within bulk-tissue constructs will usually not remain viable.

A further aspect of the invention is a structure produced by a method as hereinbefore described.

Preferred structures are those containing a network of perfusable channels, preferably a microfluidic network, produced by an above-described method.

Furthermore, a network of perfusable channels in such preferred structures can comprise inlet and outlet channels that are connectable with an inlet and an outlet of a perfusion chamber. In such a perfusion chamber, the inlet and the outlet can be connected to a pumping device, such as a peristaltic pump or a syringe, in order to maintain a convective flow across the network.

In one embodiment, such a structure containing living cells can be a network with a top side and a bottom side, wherein the living cells are adhered to the channel walls in a non-uniform distribution. The network is characterised in that the cell density is higher in the portions of the channel walls facing the bottom side than in the portions of the channel walls facing the top side.

A further aspect of the present invention relates to a method for aligning and synchronising multiple dispensing units, preferably in a method as hereinbefore described. In such an aligning and synchronizing method, a multi-component test-pattern is deposited and assessed. In an even more preferred embodiment, the multi component test-pattern comprises arrayed lines that are preferably obtained by depositing single dots with different dispensing units. Furthermore, the multi-component test-pattern can be imaged, in particular microscopically. The quality of the alignment can be evaluated by image analysing techniques, in particular by linear interpolation methods. This allows for a very precise alignment of the dispensing units according to pre-set criteria:
The required quality of interpolation (R2) can be higher than 0.999.
The required inclination variability can be lower than 0.25°.
The required pattern derivation of the trend-line can be shorter than 10 μm.

Furthermore, ejection speed and droplet diameter can be adjusted by printing parameter tuning. In cases wherein the sacrificial layers and permanent layers are deposited through a thermal or piezoelectric ink jet technique, the parameters tuned can be selected from the list comprising of voltage, pulse length and frequency.

A further aspect of the invention is a method of providing an artificial tissue using a structure as hereinbefore described as a template. Thus a structure containing living cells according to the present invention can be used to provide an artificial tissue. In preferred methods, an artificial tissue can be selected from the list comprising brain tissue, skin tissue, ocular tissue, muscular tissue, pulmonary tissue, cardiac tissue, venous tissue, artery tissue, lymphoid tissue, mammary tissue, thymus tissue, stomach tissue, liver tissue, pancreatic tissue, intestinal tissue, kidney tissue, bladder tissue, cartilage tissue, tendon tissue, bone tissue.

A further aspect of the invention is an artificial tissue produced by a method as hereinbefore described. Preferred artificial tissues can be selected from the list comprising brain tissue, skin tissue, ocular tissue, muscular tissue, pulmonary tissue, cardiac tissue, venous tissue, artery tissue, lymphoid tissue, mammary tissue, thymus tissue, stomach tissue, liver tissue, pancreatic tissue, intestinal tissue, kidney tissue, bladder tissue, cartilage tissue, tendon tissue, bone tissue.

Further advantages and features of the present invention become apparent from the following description of several embodiments and from the figures.

### DETAILED DESCRIPTION OF THE INVENTION

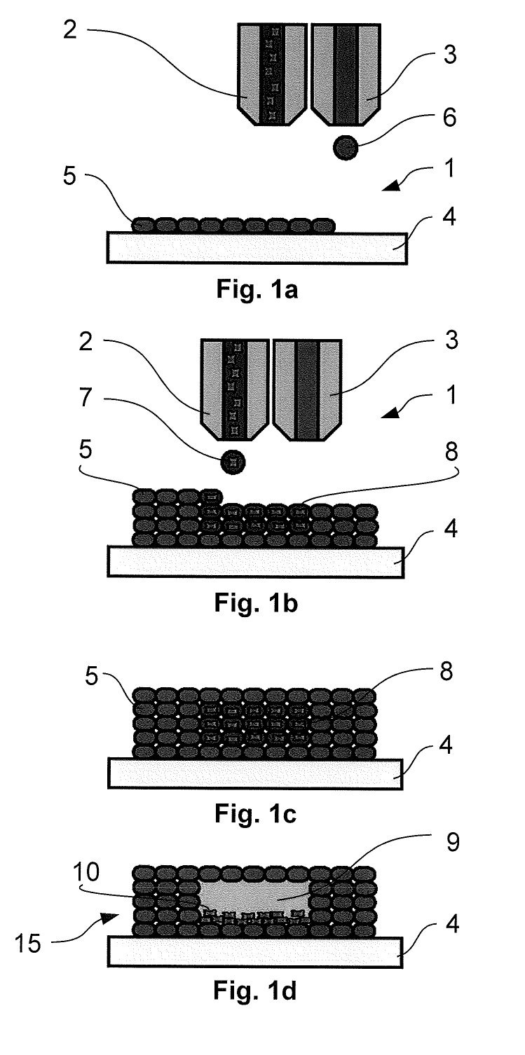

FIGS. 1a-d show a schematic representation of a method for building a structure containing living cells by droplet-by-droplet deposition according to the present invention. The device 1 of carrying out this method comprises a first dispensing unit 2 for dispensing a first composition and a second dispensing unit 3 for dispensing a second composition. In FIG. 1a, a first permanent layer 5 is dispensed on a substrate layer 4 by dispensing the second composition in form of droplets 6. FIG. 1b shows the same deposition process at a later stage. In addition to the permanent layers 5, sacrificial layers 8 have been dispensed from the first dispensing unit 2 in form of droplets 7. In FIG. 1c, the dispensing process is completed with the sacrificial layers 8 entirely covered by permanent layers 5.

Figure ld shows the finished structure containing living cells. It can be seen that the sacrificial layers 8 have been removed to provide a hollow space 9. In the course of this degradation process, the living cells 10 have been liberated and sedimented to the bottom face of the hollow space 9, in order to provide a non-uniform cell distribution.

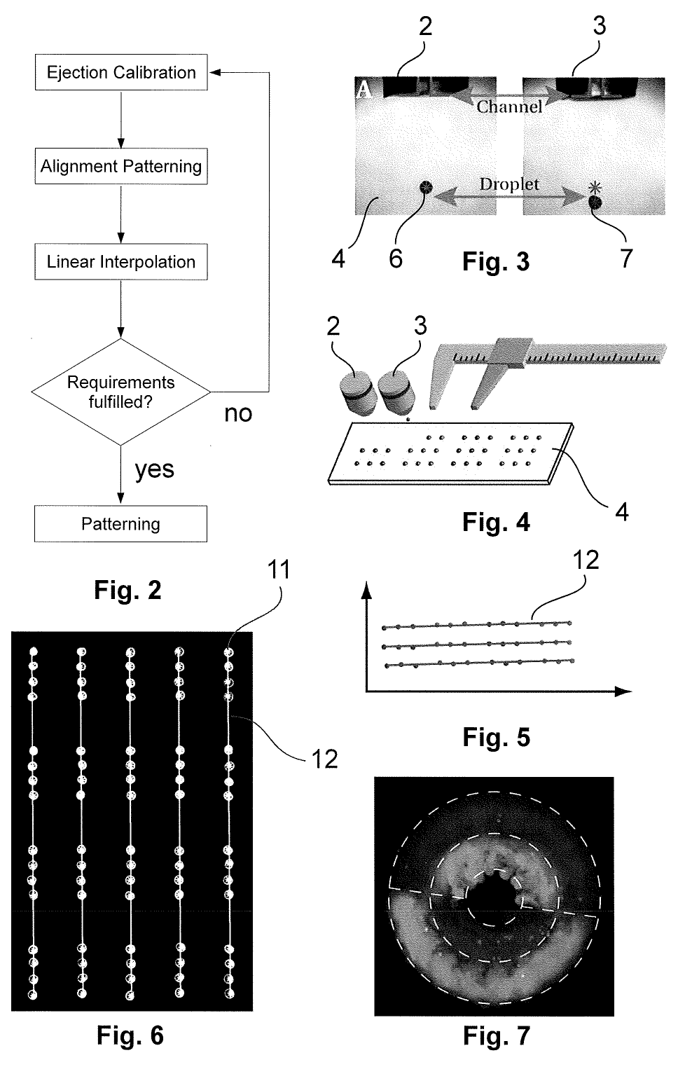

FIG. 2 shows a flow chart of a method for aligning and synchronizing multiple dispensing units according to the present invention. The method commences with ejection calibration of the dispensing apparatus. In this step, the dispensing units are aligned and the dispensing parameters are tuned, such that a desired droplet volume is dispensed at the right location. In the following alignment patterning step, a multiple-component test-pattern is deposited. This test pattern is then subjected to a linear interpolation step, in which the pattern is assessed. Criteria for this assessment can be the quality of interpolation (R²), the inclination variability or the pattern derivation from a trend-line. After termination of these parameters, the decision is taken, if pre-set requirements are fulfilled in order to start the patterning. If this is not the case, the sequence has to be recommenced at the ejection calibration step. Requirements for the interpolation parameters can be, for instance, a quality of interpolation (R²) of 0.999 and inclination variability of less than 0.25° or a pattern derivation that is shorter than 10 μm.

FIG. 3 illustrates a stage of the ejection calibration step in FIG. 2. After recording the positions of the dispensing units 2 and 3 (in the present case ink jet dispensers) with a specialized camera and calculating their relative positions, their operation parameters are tuned such that an equal diameter of the droplets 6 and 7 is achieved at identical ejection speeds.

FIG. 4 shows a schematic example of an alignment patterning step according to scheme 2. In this step, a multiple component test-pattern is deposited on the substrate layer 4 with both dispensing units 2 and 3. In the shown example, the test pattern comprises arrayed lines that are obtained by depositing single dots with different dispensing units. After deposition, the alignment pattern is imaged and the resulting image is processed by image processing techniques.

FIG. 5 shows a schematic example of a linear interpolation according to scheme 2. It can be seen that several lines 12 are laid through the alignment pattern.

FIG. 6 shows the processing of an imaged alignment pattern. The locations of the single dots 11 of the pattern are determined by an image processing technique and interpolation lines 12 are laid over the dot pattern.

FIG. 7 shows a test pattern that has been produced by the deposition of two different components using multiple deposition units that have been aligned and synchronized according to the described method.

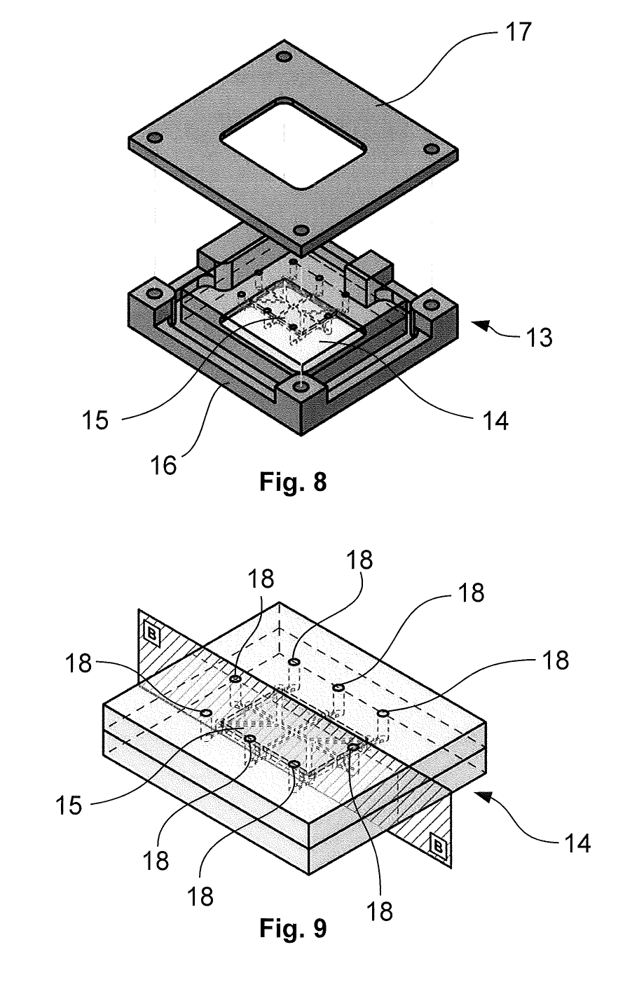

FIG. 8 shows a perfusion chamber 13 for a structure 15 containing living cells according to the present invention. The structure 15 is held in a perfusion system 14. This perfusion system 14 resides in a frame 16 with a cover 17.

FIG. 9 shows a detailed representation of a perfusion system 14 according to FIG. 8. It can be seen that the perfusion system 14 comprises several ports 18 that can serve as inlets or outlets, respectively, in order to connect the perfusion system to external devises. For instance, such an external device can be a peristaltic pump or a syringe, to establish a constant perfusion flow.

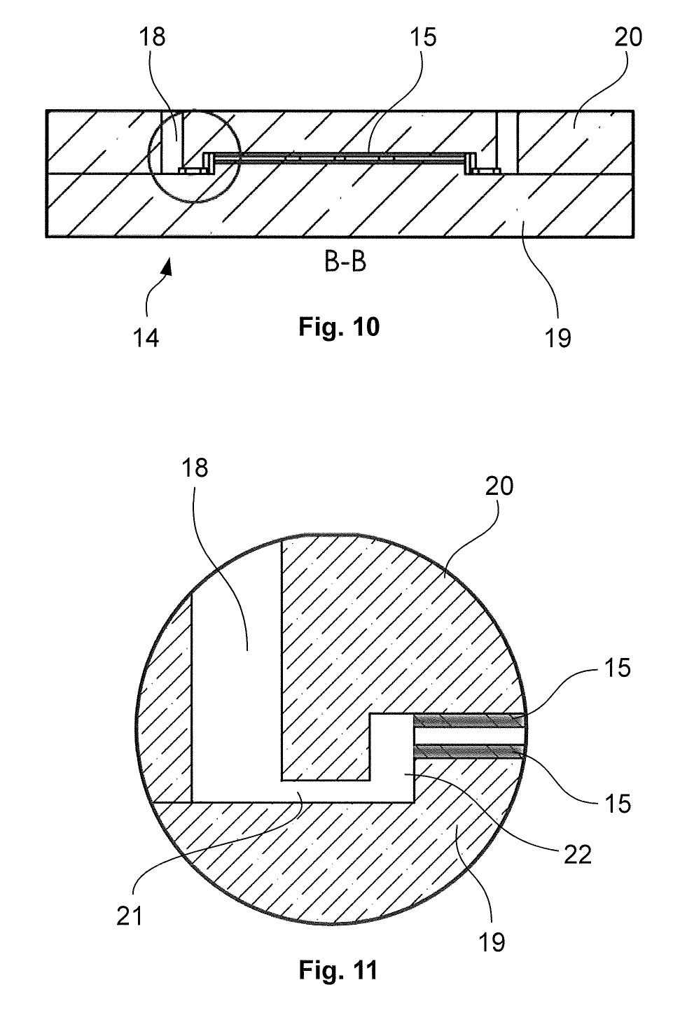

FIG. 10 shows a cross section through the perfusion system 14. It can be seen that the perfusion system 14 comprises a bottom plate 19 and a top plate 20, in between which the structure 15 is arranged.

FIG. 11 provides a sectional enlargement of the circled area in FIG. 10. It can be seen that the port 18 in the top plate is connected to the structure 15 through the channels 21 and 22.

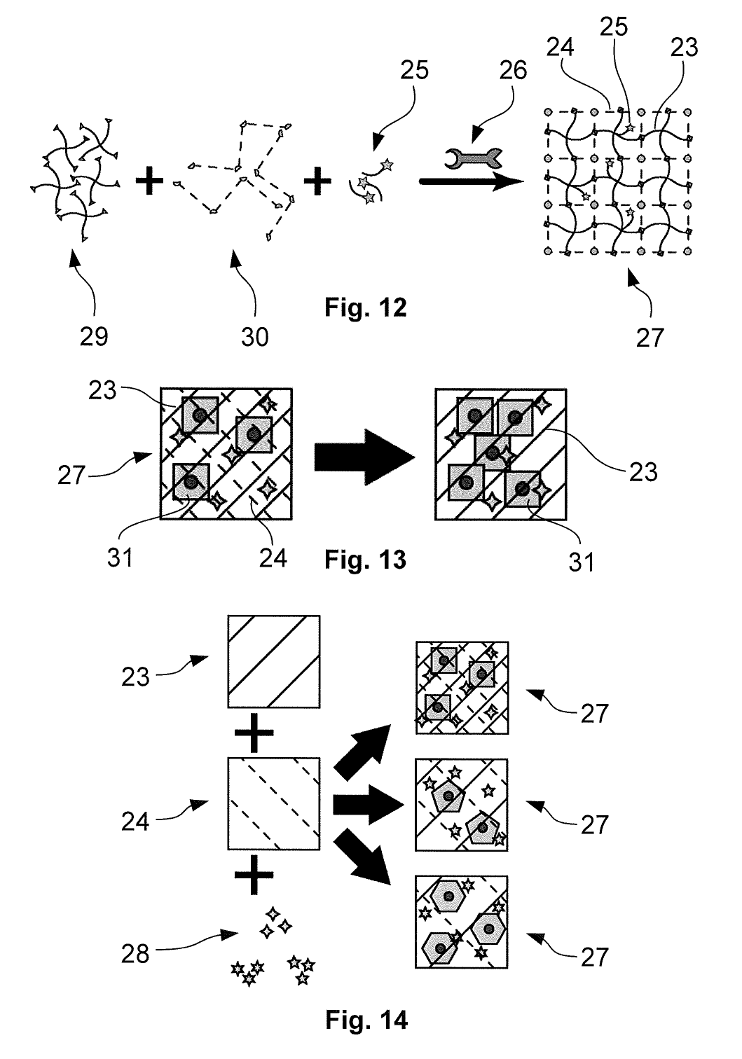

FIG. 12 provides a schematic overview of the preparation of a hybrid hydrogel network 27. The network 27 is composed of two independently curable hydrogel networks, namely the principal matrix 23 and the modulator matrix 24. The shown hybrid hydrogel system is designed in such a way that only the principal matrix 23 is utilised to attach additional factors 25, in particular biologically active moieties such as extracellular matrix components or growth-factors, such as to render the hybrid gels specifically functional towards certain cell types or control a certain cell function. The modulator matrix 24, in this case alginate, has only a transient mechanical functionality in this hydrogel system. As such, after completion of cross-linking of the precursors 29 and 30 through the cross-linker 26, the modulator matrix 24 can be selectively removed by degradation in order to generate a more permissive matrix. The precursors of the principal matrix 29 and the modulator matrix 30 can be mixed in different ratios, in order to target specific hydrogel features and biological functions. The additional biological active factors 25, such as growth factors or peptide adhesion ligands or regions can be added to the pre-cure mixture in order to tailor the microenvironment toward specific cell types and cell functions. When calcium is added to the mixture, it triggers cross-linking of both networks. In the present system, the cross-linking of alginate occurs in a timeframe of milliseconds to seconds and precedes the later cross-linking of polyethylene glycol, which proceeds within minutes.

The concept of selective degradation of the modulator matrix 24 is outlined in FIG. 13. The selective degradation of a modulator matrix 24 facilitates a better biological performance of the encapsulated cells 31, because it enhances the physiological functionality of the principal matrix 23. Specifically a modulator matrix 24 can be used to generate defects within a hybrid matrix 27, in order to promote three-dimensional cell migration, proliferation, self-renewal and differentiation. Moreover, the hybrid chains can be dramatically modified by targeting just one modulator network.

Specifically, alginate can be selectively cleaved using an enzymatic degradation strategy, preferably via alginate lyases. This allows modifying the overall stiffness of the hybrid matrix 27 without affecting the key biochemical properties of the principal matrix 23. This strategy is crucial to control cell behaviour within the hybrid matrix 27 at a desired time point. Indeed, alginate removal leads to an overall softening of the principal matrix 23 and to the introduction of defects within the hydrogel matrix. Such microenvironmental changes lead to much improved cell proliferation and three dimensional migration. As an additional beneficial effect, the degradation of alginate enables cells to interact more with the bioactive network (principal matrix 23), which results in more pronounced biological effects.

As schematically outlined in FIG. 14, the merging of the principal matrix 23 with the modulator matrix 24 into a hybrid hydrogel system 27 can enable the formulation of cellular microenvironments that recapitulate the dynamic mechanical and biochemical properties of cellular microenvironments in the human body. On one hand, a multitude of biologically active molecules 28 can be incorporated into a poly(ethylene glycol)-based network in order to mimic native cellular microenvironments. On the other hand, the mechanical properties of the hybrid gels can be tuned by modifying the polymer concentrations, molecular weight and the functionality of both naturally derived alginate and synthetic polymers. The two components can be independently modified in order to precisely and dynamically target the stiffness of a cellular microenvironment of interest. This strategy results in a material, in which the principal matrix 23, the modulator matrix 24 and additional factors can be tuned independently of one another and combinatorial. To better match biological requirements of specific cell times that are encapsulated.

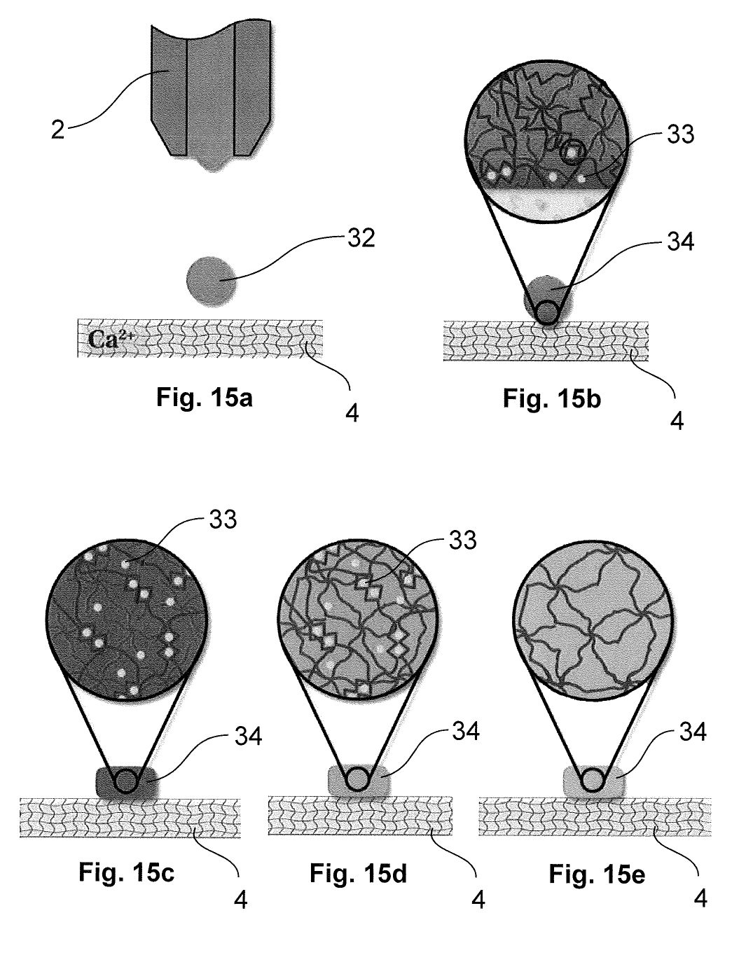

FIGS. 15a-e provide a schematic representation of an application of the alginate-PEG-based hybrid hydrogel system in droplet deposition. In FIG. 15a, a droplet 32 comprising an above-described precursor mixture is deposited on the substrate layer 4 by the dispensing unit 2. In the present case, the droplet contains 0.5% alginate and 3% TG-PEG. FIG. 15b represents the first stage after deposition with a partial enlargement of the contact area between the dispensed droplet 34 and the substrate layer 4. It can be seen that the Ca²⁺ ions 33 diffuse from the substrate layer 4 into the dispensed droplet 34 to induce gelation. Upon exposure to Ca²⁺, rapid cross-linking of the alginate portion of the hydrogel network occurs. In FIG. 15c, cross-linking for the alginate is substantially complete, but the PEG cross-linking has not reached completion yet. In FIG. 15d, the PEG cross-linking reaction has also reached substantially full conversion and the hybrid hydrogel network is fully established. FIG. 15e shows the PEG-based hydrogel droplet after removal of the alginate matrix.

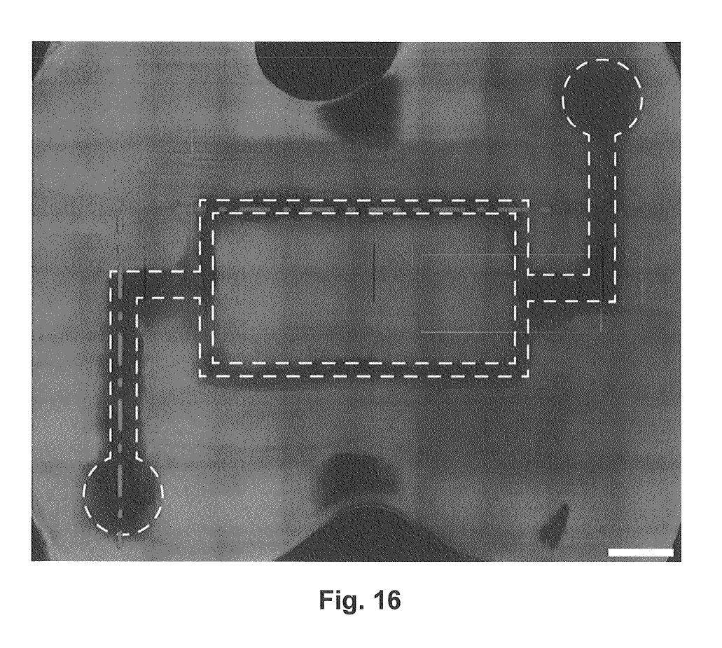

FIG. 16 represents a longitudinal section of a printed micro-fluidic network. Confocal microscopy allowed ensuring proper connectivity across the entire printed network. The permanent ink is represented in green, while the sacrificial layer was previously removed from the structure. Scale bars 1 mm.

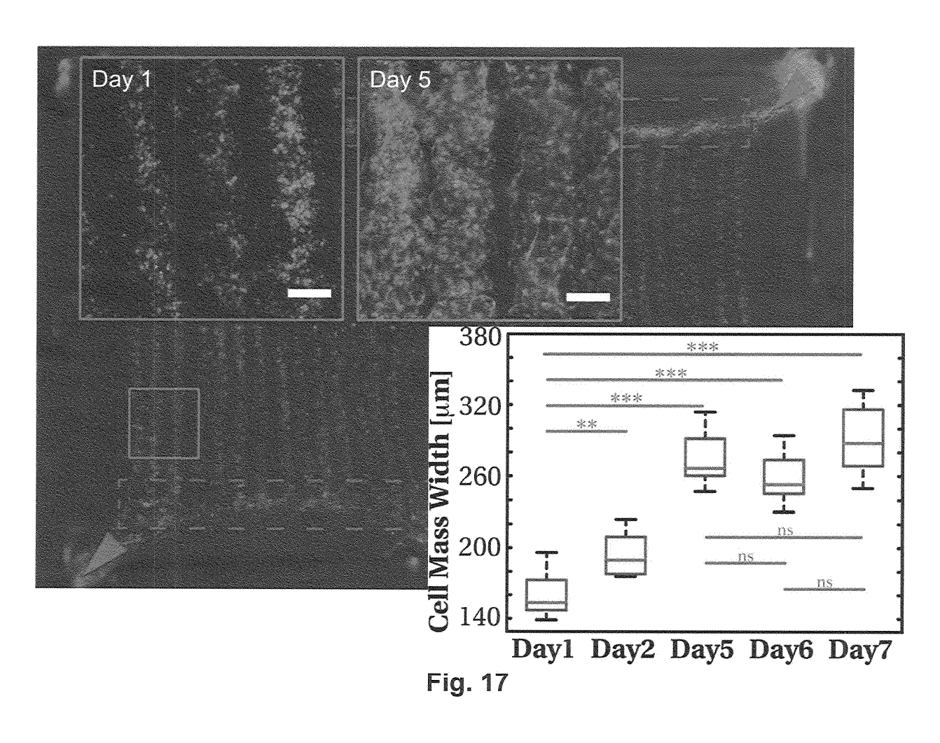

FIG. 17 represents a full example of the described bioprinting method. A hybrid hydrogel was used as permanent matrix. Cells were dispersed in the sacrificial material, specifically alginate. In the first 5 days of culture, we observed a significant increase in cell mass, indeed cells covered completely the empty space left from sacrificial material removal. The graph on the right represents the evaluation of cell growth along the first week of culture upon printing and sacrificial material removal.

### Materials

All reagents, if not otherwise mentioned, were purchased from Sigma-Aldrich AG (Bucks, Switzerland).
The Autodrop platform (Microdrop GmbH, Norderstedt, Germany) was used as the robotic dispenser. It was equipped with a MD-K-130 nozzle characterized by an outlet diameter equal to 70 μm.
Data analysis was performed either using Excel (Microsoft, Redmond, USA) or Matlab (Mathworks, Natick, USA), which was also used for image analysis.

### Preparative Example of Hydrogel Precursor Preparation

This example is based on PEG FXIIIa (3% w/v) mixed with alginate.

The alginate was added, in 0.5% w/v concentration, to transglutaminase blend as described in Biomacromolecules, vol. 8, pp. 3000-3007, October 2007, introducing three main variations: i) calcium was removed, ii) EDTA (0.66 mM) was added, and iii) total enzyme concentration was increased to 30 U/mL. Aliquots of 100 μL DN gel precursor solution were prepared by combining 18.75 μL PEG solution (13.33% w/v stock), 25 μL alginate solution (2% w/v stock), 9.09 μL tris(hydroxymethyl)aminomethane buffer solution (TBS 11x stock), 0.33 μL EDTA (200 mM stock), 31.83 μL water and 15 μL FXIIIa solution (200 U/mL stock).

Modified polyethylene glycols were prepared as set out in Biomacromolecules 2007, 8, 3000-3007.

### Preparative Example of Hydrogel Fabrication

The absence of calcium was handled by casting or printing the gel precursor solution onto calcium releasing substrate (gelatin 2% supplemented with 1% agarose), allowing for cross-linking to occur. The hydrogel substrate was prepared by dissolving 0.2 g of gelatin and 0.1 g of agarose in 20 mM calcium chloride and 0.9% sodium chloride solution (10 mL). The substrate was cross-linked by boiling the blend and then casting it in a mold and allowing it to cool. Cross-linking of the hydrogel precursor solution was performed differently for hand-casted and printed samples; hand-casted gels were left cross-linking at room temperature for 5 minutes followed by 45 minutes in controlled atmosphere (37° C., 100% humidity and 5% CO₂), while printed samples (room temperature) were immediately moved following deposition to a cell culture environment for 15 or 30 minutes.

### Example of Selective Removal of Alginate from Hydrogel Structures

Unmodified alginate was removed from the final hydrogel by a two-step method. After PEG cross-linking, samples were immersed in alginate lyase solution (1 U/ml) for two hours at 37 ° C. in controlled atmosphere (100% humidity and 5% CO₂). After two washes with 1× PBS, the samples were immersed in 10 mM EDTA for 1 hour and kept in cell culture conditions (37° C., 100% humidity and 5% CO₂). After further washing, the samples were stored/cultured in appropriate conditions.

### Cell Culture

Red fluorescent fibroblasts were cultured in Dulbecco's Modified Eagle Medium to which we added 10% fetal bovine serum, 1% penicillin/streptavidin and 50 mM HEPES. Culturing flasks were stored at 37° C., 100% humidity and 5% CO₂.

### Example of Bioink Preparation

To prepare the bioink, 1E6 cells/mL were added to the gel formulation. The cell solution was prepared as follows. Fibroblasts were washed twice with 1× PBS, and were then incubated with trypsin for five minutes. Adding cell culture medium then blocked enzyme action. The suspension was then moved in a conical tube and cells were spun down (1300RPM, for 5 minutes). The supernatant was removed and the cells were re-suspended (6E6 cells/mL) in lx PBS containing 1.5 mM EDTA. The bioink was obtained following the aforementioned protocol, but part of the water was substituted with cell suspension (16.67 μL) and Lysine-RGD ligand (final concentration 50 mM). As control, we prepared alginate-based bioink: alginate 0.8% w/v containing 1E6 cells/mL.

1. A composition comprising a first material and a second material, wherein said first material is cross-linkable by a first cross-linking reaction and said second material is cross-linkable by a second cross-linking reaction, wherein said first cross-linking reaction and said second cross-linking reaction are inducible by a common activator.

2. A composition as claimed in claim 1, wherein said first material is a polymer, preferably a biopolymer or wherein said first material is a polypeptide, a polysaccharide or a polynucleotide, wherein preferably said first material is a polysaccharide, preferably a polysaccharide derived from alginate.

3-4. (canceled)

5. A composition as claimed in claim 1, wherein said second material comprises a biocompatible synthetic or semi-synthetic polymer wherein preferably said second material comprises a hydrophilic polymer or a swellable polymer, and wherein preferably said second material comprises a copolymer of a hydrophilic polymer and an oligopeptide, wherein preferably said oligopeptide is or provides a substrate for said second cross-linking reaction, and wherein preferably said oligopeptide is or provides a substrate for at least one additional reaction, e.g. an enzymatic reaction.

6-10. (canceled)

11. A composition as claimed in claim 1, wherein said second material further comprises an additional biomolecule having a biological function, preferably a signalling molecule e.g. a cell-adhesive peptide or a growth factor.

12. A composition as claimed in claim 1, wherein said second material comprises a modified or unmodified polyglycol, preferably wherein said second material comprises a modified or unmodified polyethylene glycol, e.q. a branched modified or unmodified polyethylene glycol.

13. (canceled)

14. A composition as claimed in claim 1, wherein said first material, when cross-linked by said first cross-linking reaction, is degradable by a first degradation reaction and said second material, when cross-linked by said second cross-linking reaction, is not degradable by said first degradation reaction.

15. A composition as claimed in claim 1, wherein said common activator is a chemical or biological agent, wherein preferably said common activator is a chemical agent or wherein said common activator is an organic compound, a metal salt or ion, an acid or a base, wherein preferably said common activator is a metal ion, preferably an alkali metal ion or an alkaline earth metal ion, or wherein preferably said common activator is a calcium ion, preferably Ca⁺ or wherein said common activator is an enzyme cofactor.

16-20. (canceled)

21. A composition as claimed in claim 1, wherein said first cross-linking reaction and said second cross-linking reaction are different reactions, and wherein preferably said first cross-linking reaction and said second cross-linking reaction occur independently of each other, and wherein said first cross-linking reaction is relatively fast, preferably in the order of about 0.1 to 10 seconds, and said second cross-linking reaction is relatively slow, preferably in the order of about 10 to 60 minutes.

22-23. (canceled)

24. A composition as claimed in claim 1, further comprising a buffer, preferably tris (hydroxymethyl) aminomethane buffer and/or further comprising a chelating agent, preferably a biocompatible chelating agent, wherein preferably, said chelating agent comprises an organic acid, e.q. citric acid, or EDTA, preferably EDTA and/or further comprising a surfactant, preferably a nonionic propylene glycol- derived surfactant and/or further comprising an enzyme, preferably a cross-linking enzyme, wherein preferably said enzyme is a transglutaminase, preferably FXIIIa, wherein preferably said enzyme mediates said second cross-linking reaction.

25-30. (canceled)

31. A composition as claimed in claim 1, wherein said first cross-linking reaction and said second cross-linking reaction proceed under mutually compatible reaction conditions, wherein preferably said mutually compatible reaction conditions are physiological conditions.

32. (canceled)

33. A composition comprising a polysaccharide derived from alginate, a modified or unmodified polyethylene glycol and a transglutaminase.

34. A composition as claimed in claim 1, comprising about 0.3 to 1.0% w/v of said first material, preferably about 0.5% w/v., preferably comprising about 2.0 to 3.5% w/v of said second material, preferably about 2.5% w/v.

35. (canceled)

36. A bioink comprising a composition as claimed in claim 1.

37. A bioink as claimed in claim 36, further comprising cells, preferably eukaryotic cells, e.g. mammalian cells.

38. A hydrogel formed from the composition according to claim 1, or a bioink as claimed in claim 36.

39. A hydrogel comprising a cross-linked first material formed by a first cross-linking reaction and a second cross-linked material formed by a second cross-linking reaction, wherein said first cross-linking reaction and said second cross-linking reaction are inducible by a common activator.

40. A hydrogel as claimed in claim 39, wherein said cross-linked first material is a polypeptide, a polysaccharide or a polynucleotide, wherein preferably said cross-linked first material is a polysaccharide, preferably a polysaccharide derived from alginate.

41. (canceled)

42. A hydrogel as claimed in claim 40, wherein said cross-linked second material comprises a biocompatible synthetic or semisynthetic polymer, wherein preferably said second material comprises a co-polymer of a hydrophilic polymer and an oligopeptide, and wherein preferably said oligopeptide is or provides a substrate for said second cross-linking reaction and wherein preferably, said oligopeptide is or provides a substrate for at least one additional reaction, e.g. an enzymatic reaction.

43-45. (canceled)

46. A hydrogel as claimed in claim 39, wherein said second material further comprises an additional biomolecule having a biological function, preferably a signaling molecule e.g. a cell-adhesive peptide or a growth factor.

47. A hydrogel as claimed in claim 39, wherein said cross-linked second material comprises a modified or unmodified polyethylene glycol, e.g. a branched modified or unmodified polyethylene glycol, and wherein preferably said cross-linked first material is selectively degradable in the presence of said cross-linked second material, wherein preferably said cross-linked first material is selectively degradable in a biocompatible process, preferably wherein said cross-linked first material is selectively degradable using an enzyme; and
wherein preferably said cross-linked second material is selectively degradable by a cell-controlled mechanism, and preferably said cross-linked second material is selectively degradable using an enzyme, preferably a cell-secreted protease, e.g. a matrix metalloprotease.

48-52. (canceled)

53. A structure formed from a composition according to claim 1 or a bioink according to claim 36.

54. A structure comprising a hydrogel according to claim 38, wherein preferably the structure comprises living cells, preferably living eukaryotic cells, e.g. living mammalian cells, wherein preferably said living cells are present in said first composition or said cross-linked first composition.

55-56. (canceled)

57. A method of making a structure according to claim 53, comprising depositing a composition according to claim 1 or a bioink according to claim 36 on a substrate, wherein said substrate provides a common activator for inducing said first cross-linking reaction in said first composition and said second cross-linking reaction in said second composition.

58. A method of making a structure, comprising the steps of: i. forming at least one, preferably a plurality of, sacrificial layer(s) on a substrate; ii. forming at least one, preferably a plurality of, permanent layer (s) on said substrate or said first sacrificial layer (s); and
wherein said at least one sacrificial layer is derived from a first composition and said at least one permanent layer is derived from at least one second composition; and
wherein said at least one sacrificial layer is formed by a first cross-linking reaction of said first composition and said at least one permanent layer is formed by a second cross-linking reaction of said second composition;
wherein said first cross-linking reaction and said second cross- linking reaction are induced by said common activator.

59. A method according to claim 58, wherein said substrate provides said common activator for inducing said first cross-linking reaction in said first composition and said second cross-linking reaction in said second composition, wherein preferably said common activator is a chemical agent, preferably an organic compound, a metal salt or ion, an acid or a base, wherein preferably said common activator is a metal ion, preferably an alkali metal ion or an alkaline earth metal ion, or wherein said common activator is a calcium ion, preferably Ca²⁺.

60-62. (canceled)

63. A method according to claim 58, wherein said at least one sacrificial layer is degradable by a reaction that does not degrade said at least one permanent layer, wherein preferably the method further comprises the step of selectively removing, in particular degrading, said at least one sacrificial layer to obtain at least one hollow space, wherein preferably said selective removal of said at least one sacrificial layer is a biocompatible process, and wherein preferably an enzyme, preferably an alginate lyase, is used to selectively remove said at least one sacrificial layer, wherein preferably said at least one hollow space forms a network, in particular a microfluidic network, of perfusable channels.

64-66. (canceled)

67. A method according to claim 58, wherein said first and/or second composition further comprises living cells, preferably mammalian cells.

68. A method according to claim 67, wherein upon the selective removal, preferably degradation, of said at least one sacrificial layer, said living cells are deposited on at least one surface of said at least one permanent layer, preferably in a nonuniform distribution and wherein preferably said at least one sacrificial layer or said at least one permanent layer is deposited by extrusion or by printing preferably by thermal or piezoelectric ink jet printing.

69-72. (canceled)

73. A method according to claim 58, wherein said depositing of said at least one sacrificial layer and said at least one permanent layer is effected by a plurality of dispensing units that are aligned and synchronized to one another.

74. A structure produced by a method according to claim 57.

75. A structure according to claim 74, wherein said network of perfusable channels further comprises inlet and outlet channels that are connectable with an inlet and an outlet of a perfusion chamber.

76. A method for aligning and synchronizing multiple dispensing units, preferably in a method according to claim 73, wherein a multi-component test-pattern is deposited and assessed.

77. A method according to claim 76, wherein said multi-component test-pattern comprises arrayed lines that are preferably obtained by depositing single dots with different dispensing units, wherein preferably the multi-component test-pattern is imaged, in particular microscopically, and the quality of the alignment is evaluated by image analyzing techniques, in particular by linear interpolation methods.

78. (canceled)

79. A method of providing an artificial tissue using a structure as claimed in claims 53 as a template, wherein preferably said artificial tissue is selected from the list comprising brain tissue, skin tissue, ocular tissue, muscular tissue, pulmonary tissue, cardiac tissue, venous tissue, artery tissue, lymphoid tissue, mammary tissue, thymus tissue, stomach tissue, liver tissue, pancreatic tissue, intestinal tissue, kidney tissue, bladder tissue, cartilage tissue, tendon tissue and bone tissue.

80. (canceled)

81. An artificial tissue produced by a method according to claim 79.
